(19) United States
(12) Patent Application Publication
(10) Pub. No.: US 2022/0080195 A1
(43) Pub. Date: Mar. 17, 2022

(54) ELECTRICAL STIMULATION SYSTEM FOR FACIAL EXPRESSIONS AND A METHOD OF USE THEREOF

(71) Applicants: Nazmi Peyman, Richmond, VA (US); Edmond Zahedi, Burnaby BC (CA)

(72) Inventors: Nazmi Peyman, Richmond, VA (US); Edmond Zahedi, Burnaby BC (CA)

(21) Appl. No.: 17/473,675

(22) Filed: Sep. 13, 2021

Related U.S. Application Data
(60) Provisional application No. 63/077,110, filed on Sep. 11, 2020.

(51) Int. Cl. A61N 1/36; A61N 1/04

(52) U.S. Cl. CPC A61N 1/36003; A61N 1/36031; A61N 1/0484; A61N 1/0452

(57) ABSTRACT

A novel electrical stimulation system to stimulate facial muscles so that face expressions mimicking the happy face of a person can be induced in a person. The system includes a series of electrodes configured on a cosmetically acceptable medium, such as a mask that can be applied on the face. The position of the electrodes and the stimulation patterns and stimulation parameters can be determined by a pre-trained artificial intelligence algorithm.

### CROSS-REFERENCE TO RELATED APPLICATIONS

This application claims priority to the U.S. provisional patent application Ser. No. 63/077,110, filed on Sep. 11, 2020, which is incorporated herein by reference in its entirety.

### FIELD OF INVENTION

The present invention relates to an electrical stimulation system, and more particularly, the present invention relates to an electrical stimulation system for facial muscles to induce artificial facial expressions.

### BACKGROUND

It is well known that emotional states in humans can manifest themselves in facial expressions. There is ample evidence that the reverse is also true: a fake or forced smile can “trick” the brain into releasing dopamine and serotonin and make a subject feel happier and feel less stressed. This two-way communication between the brain cortex and the facial muscles can also be effective during sub-threshold stimulation.

A variety of ways are known for inducing smiles for a happy mood in humans: laughing exercises in groups, mechanically stretching the face to make appear a “smiley”, or even holding an object such as a pencil in the mouth until the facial expression appears.

However, the known methods of inducing smiles are mechanical and unreliable. A need is appreciated for a device that can induce desired facial expressions based on predefined algorithms in humans. A need is appreciated for determining suitable facial expressions in humans and inducing the same through an external device.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for inducing artificial facial expressions in humans.

It is another object of the present invention that the induced facial expressions can elicit mood and trigger the release of desired chemical messengers by the brain.

It is still another object of the present invention that the inducing of facial expressions can be automated.

In one aspect, disclosed is a system and method of inducing artificial facial expressions in humans by a series of electrodes to deliver electrical stimulations to the facial muscles involved in the making of the facial expressions. Preferably, the facial expressions can be a smile that can trigger the brain to release mood-elevating chemical messengers.

In one aspect, a dynamic face model can be created using imaging technologies from a face that can include information such as face morphology, anatomy, muscle tone, existing facial expressions, and like details of a face. This dynamic face model can be used to generate a 3-D map that can define the location of electrodes on the face for inducing a facial expression. The dynamic face model can also include information for defining a stimulation pattern for electrodes.

In one aspect, cameras and sensors can be used to generate the dynamic face model. In one case, one or more cameras can be used to capture or more images of the face. In one case, the sensor can detect movements of the facial muscles. In one case, the sensors can detect the electrophysiology of the facial muscles. In one case, the sensors can be muscle tone sensors. In one case, the sensors can include gyroscopes and accelerometers to detect facial muscle movement.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 depicts a neutral facial expression of a subject when no electrode mask is used, according to an exemplary embodiment of the present invention.

FIG. 2 depicts the electrode mask, according to an exemplary embodiment of the present invention.

FIG. 3 depicts an electrode mask applied to a face to stimulate the Zygomatic, buccal, and parotid-masseteric muscles, according to an exemplary embodiment of the present invention.

FIG. 4 depicts another electrode mask applied to the face to stimulate the frontal set of muscles, according to an exemplary embodiment of the present invention.

FIG. 5 depicts another electrode mask applied to the face to stimulate the supraorbital muscles, according to an exemplary embodiment of the present invention.

FIG. 6 depicts another electrode mask applied to the face to stimulate the infraorbital muscles, according to an exemplary embodiment of the present invention.

FIG. 7 depicts another electrode mask applied to the face to stimulate the temporal muscles, according to an exemplary embodiment of the present invention.

FIG. 8 depicts electrode masks applied to the face to stimulate a set of facial muscles for inducing the desired facial expression, according to an exemplary embodiment of the present invention.

FIG. 9A depicts steps of a disclosed method in the generation of the dynamic face model and the electrode mask, according to an exemplary embodiment of the present invention.

FIG. 9B depicts the disclosed system for inducing artificial facial expressions in humans, according to an exemplary embodiment of the present invention.

FIG. 10A shows steps in the generation of the electrode mask, according to an exemplary embodiment of the present invention.

FIG. 10B depicts real-time control and feedback loop in the electrical stimulation of the facial muscles inducing a facial expression, according to an exemplary embodiment of the present invention.

FIG. 11 depicts a series of implanted electrodes (below the skin) to stimulate a targeted group of facial muscles.

FIG. 12 is a block diagram showing a system architecture, according to an exemplary embodiment of the present invention.

FIG. 13 is a flow chart showing a method for electrical stimulation, according to an exemplary embodiment of the present invention.

### DESCRIPTION OF EMBODIMENTS

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

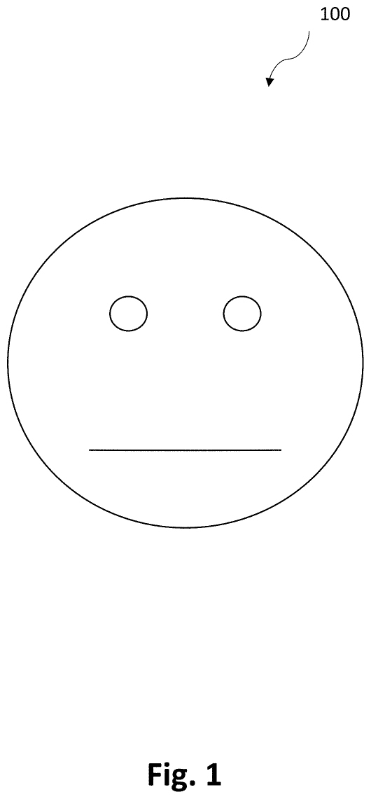

Disclosed is an electrical stimulation system for facial muscles and a method for inducing artificial facial expressions in humans. It is envisioned that the artificial induction of certain facial expressions, such as a smile, using the disclosed system can trigger the brain to release mood-elevating chemical messengers. Referring to FIG. 1 which shows a face 100 with a neutral expression. Such a facial expression can be a manifestation of depression or similar mind status of a subject. To elevate the mood of the subject, the disclosed system can artificially induce certain facial expressions by electrically stimulating a set of facial muscles. The artificially making of the happy facial expressions can trigger the brain to release mood-elevating chemical messengers, thus making the subject happier in real.

Thus, the electrical stimulation of the facial muscles to induce artificial expressions can be referred to as an electrostimulation therapy for elevating mood in a subject. The disclosed system can determine different parameters, such as intensity, waveforms, and frequencies for the electrostimulation therapy. Generally, facial expressions are a result of the complex cooperation of a set of facial muscles. The Disclosed system using artificial intelligence-based algorithms can learn from the face anatomy and optionally electrophysiology of the facial muscles to create a 3D map for a subject that can define the positioning of different electrodes and a stimulation pattern for delivering the electrical stimulation by the distributed electrodes to targeted facial muscles. In one case, stimulations can also be of a sub-threshold intensity that can trick the brain to release the chemical messengers without actual muscle contraction.

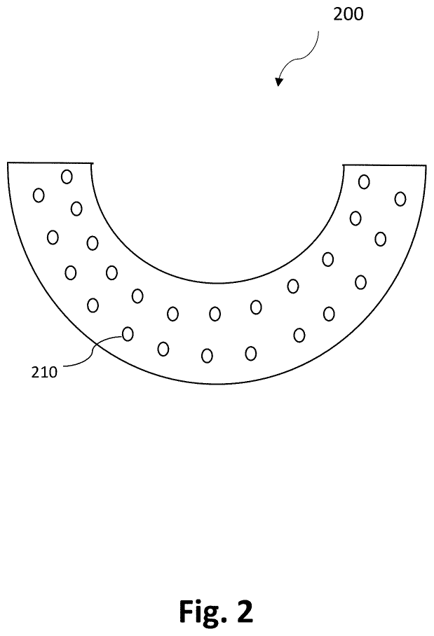

In one exemplary embodiment, a set of electrodes can be strategically positioned on the face to stimulate a set of target facial muscles in a pre-programmed order to induces the respective facial expression. The set of electrodes can also be incorporated in an electrode mask 200 having multiple electrodes 210 that can be applied to the face for stimulating respective facial muscles. FIG. 2 shows one such exemplary embodiment of the electrode mask. To be effective, the electrode mask may be flexible that can conform to the contours of the face. Moreover, it is also envisioned that the electrode mask can also be aesthetically acceptable. The shape and size of the electrode mask can depend upon the distribution of the muscles group that the electrode mask has to target for electrical stimulation.

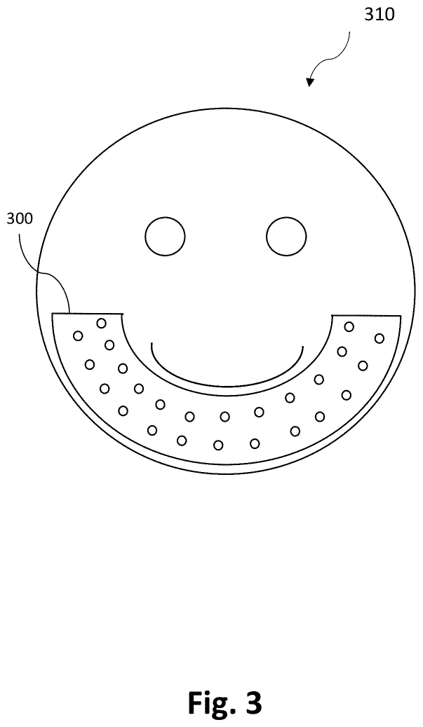

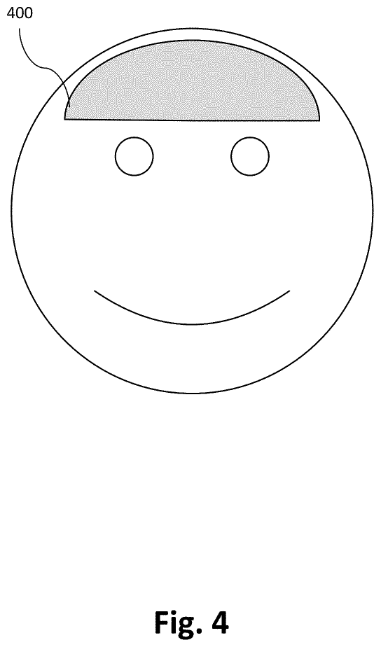

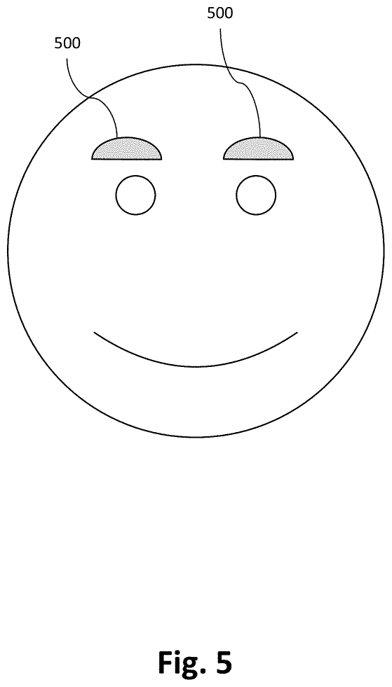

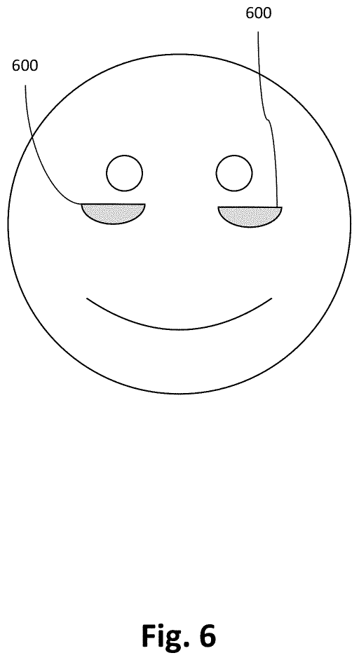

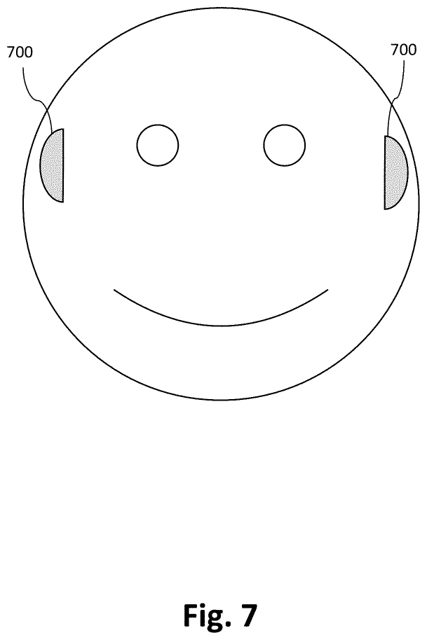

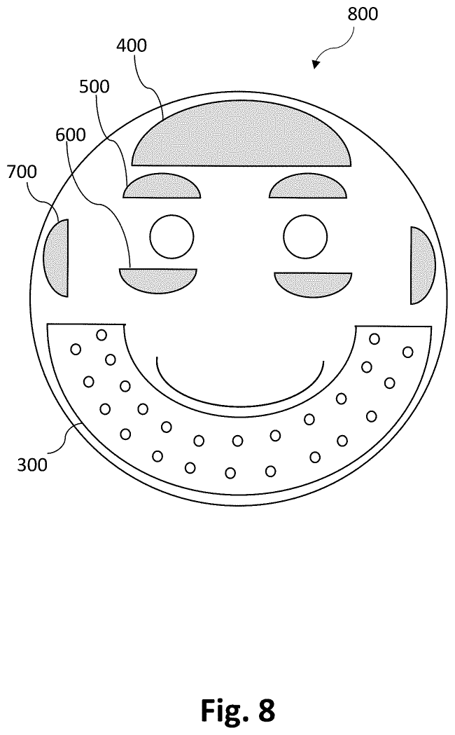

FIG. 3 shows an electrode mask 300 applied to a face 310 for electrical stimulation of a group of facial muscles: zygomatic, buccal, and parotid-masseteric. The electrical stimulation of these muscles can induce an artificial smile in the subject's face. FIG. 3 also shows the induced smile in the subject as compared to the neutral face shown in FIG. 1. Referring to FIG. 4 which shows another electrode mask 400 that can stimulate the frontal group of facial muscles. The electrical stimulation of these muscles can contribute to inducing the smile. Similarly, FIG. 5 shows two-electrode masks 500 for the supraorbital group of facial muscles, wherein the electrical stimulation of these muscles can also contribute to inducing the smile. FIG. 6 shows two-electrode masks 600 for the infraorbital group of facial muscles, where the electrical stimulation of these muscles can also contribute to inducing the smile. Another two-electrode masks 700 are shown in FIG. 7 for the temporal group of facial muscles, where the electrical stimulation of these muscles can also contribute to inducing the smile. The mask shown in FIG. 4-7 can be different in shape and size such as to juxtapose to different contours of parts of the face. Multiple electrode masks can be used simultaneously to induce a facial expression, such as a smile. For example, FIG. 8 shows the multiple electrode masks applied to the face 800, wherein the types of electrode masks that can be used together can depend upon the intended application, such as inducing an artificial smile in the subject. It is to be understood that the drawings show exemplary embodiments of the electrode masks and their arrangement, however, different electrode masks can be provided and arranged in any order for a desired facial expression without departing from the scope of the present invention.

Although the anatomy of the face and the facial muscles may be the same in humans, however, the morphology of the face differs. Moreover, the rigidity of few muscles may be different from others in different humans. Also, the skin rigidity may vary in same person at in different parts of the face, or between different persons, or in the same person in different situations. Thus, the present invention envisions a personalized electrostimulation therapy. The disclosed system can generate a dynamic face model of a subject's face using a series of images captured by one or more cameras. It is to be understood that the imaging techniques based on images and lasers are known in the art for generating 3D models of a face, such as in 3D printing, and any such technique for generating the dynamic face model is within the scope of the present invention. The disclosed system can include an artificial intelligence algorithm that can be trained to identify muscles movements and positions of the muscles in the face. For example, the artificial intelligence learning algorithm can be trained using a labeled training dataset having images of faces from several persons and images of different facial expressions from the same person in several persons. The artificial learning algorithm can be used to generate the dynamic face model from the input data from the camera and optionally sensors. In one case, images of a neutral face of the subject can be captured and can be processed by the artificial learning algorithm. In another case, the subject can be asked to create different facial expressions, such as neutral, sad, happy, angry, etc. and a series of such images can be used as an input for generating the dynamic face model. It is to be understood that the artificial learning algorithm can be trained to determine different facial expressions from the neutral face. Besides, imaging techniques, sensors can also be used to determine muscle positioning and movements. For example, sensors, such as EMG muscle sensors to detect the electrophysiology of muscles can be used to detect contractions in the muscles. Similarly, muscle tone sensors can also be used. The muscle tone sensors can also be employed to measure the amount of existing stretch in each group of facial muscles. These muscle tone sensors can provide information addition to the information acquired from facial images, and hence can be used on its own for programming the system or in conjunction with the camera/scanner data. Gyroscope and accelerometer also be used to detect muscle movements. The artificial intelligence algorithm using the inputs from the images and optionally from the sensors can generate the dynamic face model.

The dynamic face model can include data, such as anatomy, subjective mood evaluation, felt emotions, a dosage of medication consumed, significant life events affecting one's state of mind related to the subject. The dynamic face model can also include a 3D map defining the positions of target muscles. The 3D map can be used to position the electrodes on the face. The 3D map also includes information on face morphology that can be used to create electrode masks that can fit over the face conforming to the contours of the face. The dynamic face model can also generate a configuration file that can include programming for the stimulation pattern and the stimulation parameters for each electrode. The configuration file can be manually modified by a technician, expert, or healthcare professional. The system can receive data from the subject including relative mood feelings, emotions, medication being taken by the subject, any event the subject engaged in, and other facts that can affect the mood of the subject. It is to be understood that the data taken from the subject can be optional, and any data related to the subject can be taken without departing from the scope of the present invention.

The dynamic face model can be used to create the electrode masks as shown in FIGS. 2-8. These masks can be applied to different parts of the face for stimulating the target muscles. The configuration file can include information such as the pattern of the electrical stimulation, waveform, frequency, intensity, and like information for the excitation of the muscles. In brief, the configuration file can include stimulation parameters for each electrode.

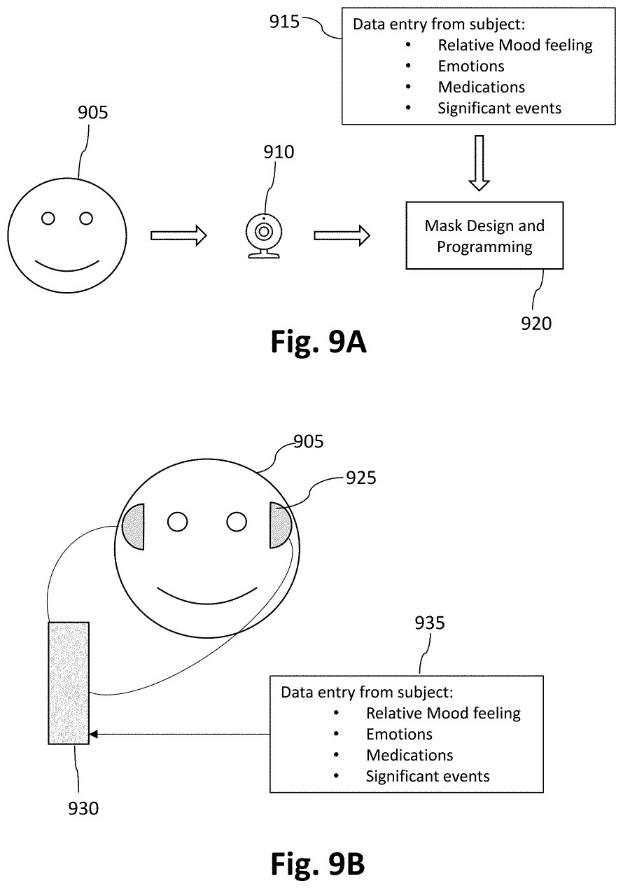

Now referring to FIG. 9A which shows steps in creating an electrode mask. shown is a camera 910 capturing images of a face 905. subject's data at block 915 can also be received. The electrode mask can be designed and programing can be written at block 920. The designing and programming can include the calibration step wherein a technician can modify the electrode mask and programming based on feedback from the trial of stimulation.

Referring to FIG. 9B which shows the implementation of the electrical stimulation system. Shown are the temporal electrode masks 925 designed at block 920 applied to the face 905. A simulator 930 can be seen coupled to the two electrode masks 925. The stimulator can deliver the stimulation pattern based on the programing at block 920 through the two electrode masks 925. Before the simulations, the system can receive the current subject's data of block 935. Based on the current subject's data, the system can choose a level of stimulation from different levels, such as low, medium, and high. For example, in case, the subject is already in a happy mood, a low level of stimulation can be delivered. Oppositely, if the subject is depressed, a high level of stimulation may be needed to achieve the desired outcome.

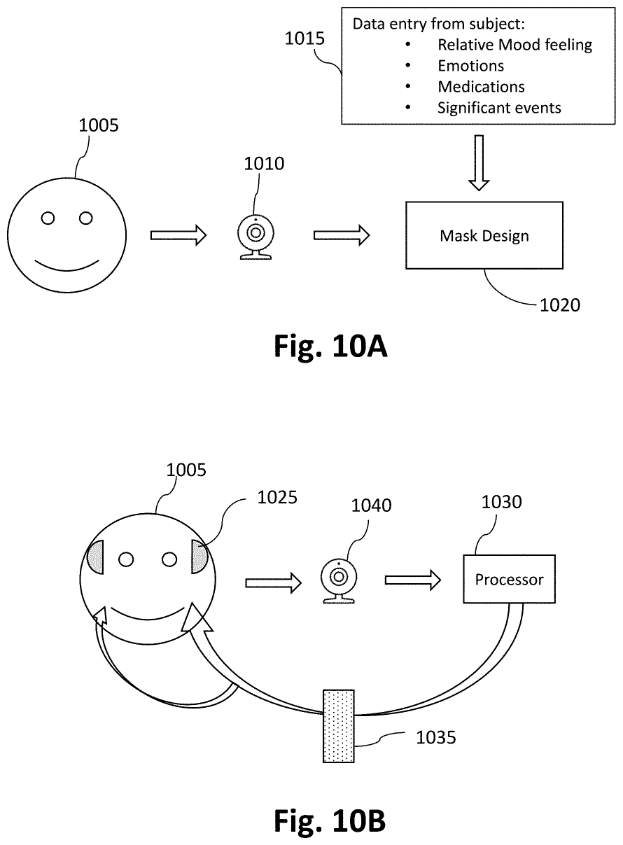

Referring to FIG. 10A which shows another exemplary embodiment of creating an electrode mask. A camera 1010 can receive images of a face 1005. subject's data at block 1015 may also be taken for designing the electrode mask at block 1020. Herein the programming may not be static but dynamically optimized based on a closed feedback loop.

Referring to FIG. 10B which illustrates the closed feedback loop. The temporal electrode masks 1025 can be applied to the face 1005. A stimulator 1035 can be connected to the two electrode masks. A camera 1040 can capture an image of the face 1005 after stimulation. The facial expressions in the image can be compared with the desired facial expression. The stimulation pattern and the stimulation parameters can be modified by the processor 1030 based on the comparison.

To adequately stimulate each electrode, the electrode mask needs to be designed and the parameters of the electrical stimulation for each electrode needs to be set. To this end, various data can be used. In one case, artificial intelligence-based algorithms can be used during the design phase for determining the location and number of electrodes. Additionally, the locations of the electrodes can be adjusted by the technician, and the artificial intelligence algorithms can further learn from the changes made by the technician. To adjust the number of electrodes, locations of electrodes, and stimulation parameters, the technician can use prior knowledge of facial muscle anatomy and/or compare the target happy image with the obtained face expression after the application of the appropriate electrical stimulation.

In one case, the relative mood feeling, emotions, medications, and significant events occurring at the time of the acquisition of the images can also be entered into the system during the design phase. These data can be used by the artificial learning algorithms to determine the necessary parameters of the electrical stimulation for each electrode, depending on the applicable circumstances. However, any intervention by the technician/operator/health professional can be optional. In the practical implementation of the stimulator, the camera/scanner can be employed to provide real-time feedback data to a processor which continuously adjusts the parameters of the electrical stimulation. The camera for feedback can be a different camera, such as an embedded camera of a smartphone or laptop. The parameters of the electrical stimulation for each electrode are sent to the stimulator in real-time which generates the signals needed by each electrode. This feedback loop allows for adjusting the electrical stimulation parameters based on the difference between the target and desired facial (happy) expressions, constantly adapting to the changing electrophysiological condition of each subject.

One advantage of the embodiment shown in FIG. 10B compared (with real-time feedback loop) to the embodiment shown in FIG. 9B may be that the parameters of the electrical stimulations are always set to their optimal values. This optimization is ensured by the feedback loop. The feedback loop ensures that the right amount of electrical stimulation reaches the electrodes creating the desired happy facial expression, without exposing the subject to unwanted stimulation.

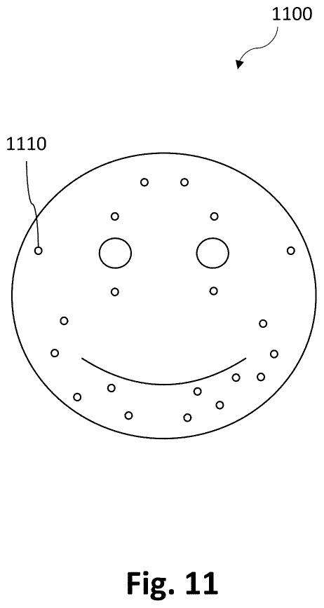

Referring to FIG. 11 which shows another exemplary embodiment wherein the electrodes are shown implanted in the targeted facial groups of muscles. The advantage of this configuration is that the subject does not have to wear a mask anymore. In this case, the embodiment as described previously in FIG. 9B (without a feedback loop) or FIG. 10B (with feedback loop) can be used before implantation during a trial period to adjust the parameters of the electrical stimulation. The location shown in FIG. 11 is the temporal area and as such, other embodiments can also be used. It is to be understood that whether the embodiment in FIG. 9B or FIG. 10 is implemented, the system can auto-validate its function based on achieved mood feeling ratings input by the user, allowing the system to perform any further adjustments.

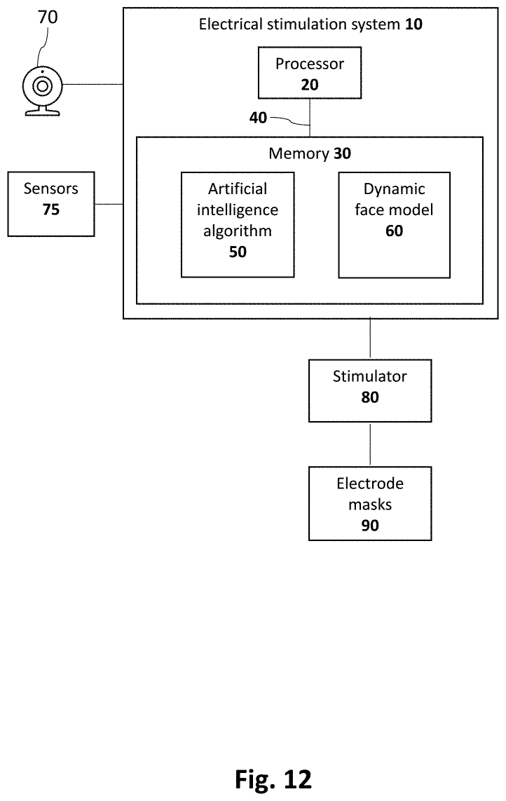

Referring to FIG. 12, which shows an architecture of the disclosed electrical stimulation system 10. The system can include a processor 20 and a memory 30 coupled through a system bus 40. The processor 20 can be any logic circuitry that responds to and processes instructions fetched from the memory 30. The memory 30 may include one or more memory chips capable of storing data and allowing any storage location to be directly accessed by the processor 20. The memory can include an artificial intelligence algorithm 50 and a dynamic face model 60. A stimulator 80 can be coupled to the electrode masks 90 through electrical wires. Also, can be seen in FIG. 12 are camera 70 and sensors 75.

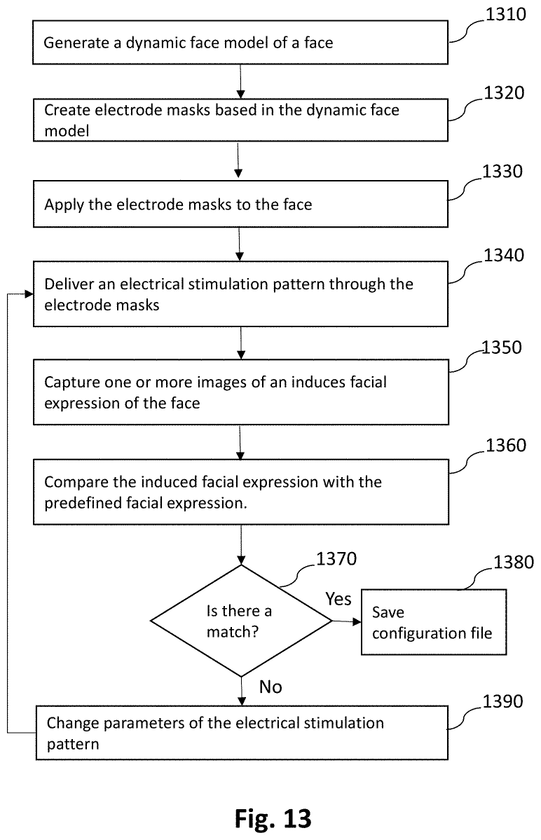

Referring to FIG. 13 which is a flow chart showing an exemplary embodiment of a method for inducing facial expressions using electrical stimulation. First, a dynamic face model can be generated at step 1310. using the dynamic face model, electrode masks can be designed, at step 1320. The electrode masks can then be applied to the face, at step 1330. upon application, the stimulator can deliver the first stimulation pattern, generated by the dynamic face model, through the electrode masks, at step 1340. An image of the face showing the induced facial expressions as a result of the electrical stimulation can be captured by a camera, at step 1350. The induced facial expression in the image can be compared by a processor with a predefined happy facial expression, at step 1360. A check can be made for the match, at step 1370. In case, there is a positive match, the configuration file can be saved, at step 1380. In case, there is a negative match, the stimulation parameters can be modified by the processor and optionally also by the technician, at step 1390. The loop 1340 to 1390 can be repeated till the positive match can be achieved.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for inducing artificial facial expressions in a user, the method comprising the steps of:
generating a dynamic face model of a face of the user, the dynamic face model comprises information related to positioning of a plurality of electrodes, a first stimulation pattern, and stimulation parameters;
applying a plurality of electrode masks to the face based on the dynamic face model, each electrode mask of the plurality of electrode masks has one or more electrodes of the plurality of electrodes; and
delivering the first stimulation pattern, based on the stimulation parameters, by the plurality of electrodes, to a set of predetermined target facial muscles inducing a first facial expression.

2. The method as claimed in claim 1, wherein the method further comprises the steps of:
upon inducing the first facial expression, capturing, by a camera, an image of the face;
comparing the first facial expression in the image with a predefined facial expression;
upon comparison, modifying the first stimulation pattern and the stimulation parameters obtaining a second stimulation pattern; and
delivering, by the plurality of electrodes, the second stimulation pattern to the set of predetermined target facial muscles inducing a second facial expression, wherein the first facial expression is different from the second facial expression.

3. The method as claimed in claim 1, wherein the first facial expression is a smile and at least one electrode mask of the plurality of electrode masks configured to stimulate zygomatic, buccal, and parotid-masseteric muscles.

4. The method as claimed in claim 3, wherein the plurality of electrode masks configured to stimulate frontal set of muscles, supraorbital muscles, infraorbital muscles, and temporal muscles.

5. The method as claimed in claim 1, wherein the set of predetermined target facial muscles comprises zygomatic muscles, buccal muscles, parotid-masseteric muscles, frontal set of muscles, supraorbital muscles, infraorbital muscles, and temporal muscles.

6. The method as claimed in claim 1, wherein the step of generating the dynamic face model further comprises the steps of:
capturing, by one or more cameras, a plurality of images of the face; and
processing the plurality of images by a pre-trained artificial intelligence algorithm to generate the dynamic face model, wherein the processing of the plurality of images comprises identifying muscles of the face and locations of the identified muscles.

7. The method as claimed in claim 6, wherein the pre-trained artificial intelligence algorithm is trained using a labelled training data comprising images of faces of different persons and images of different facial expressions of a person.

8. The method as claimed in claim 6, wherein the method further comprises the steps of:
receiving muscle data from a muscle tone sensor applied to the face, wherein the muscle data is also used by the pre-trained artificial intelligence algorithm to generate the dynamic face model.

9. The method as claimed in claim 6, wherein the method further comprises the steps of:
receiving a muscle electrophysiology data from an electromyography sensor, wherein the muscle electrophysiology data is also used by the pre-trained artificial intelligence algorithm to generate the dynamic face model.

10. The method as claimed in claim 1, wherein the method further comprises the steps of receiving a user data of the user, the user data comprises a mental state of the user, wherein the first stimulation pattern is further based on the user data.

11. The method as claimed in claim 10, wherein the user data further comprises details of medication taken by the user.

12. The method as claimed in claim 1, wherein the dynamic face model further comprises information related a face morphology, wherein the plurality of electrode masks is made based on the face morphology, wherein the plurality of electrode masks are flexible and configured to conform to contours of the face when applied to the face.

13. An electrical stimulation system comprising:
a stimulator for delivering a stimulation pattern to target facial muscles through a plurality of electrodes;
a plurality of electrode masks, wherein each electrode mask of the plurality of electrode masks has one or more electrodes of the plurality of electrodes, the plurality of electrode masks configured to be applied to a face of a user, wherein the plurality of electrode masks is further configured to conform to contours of the face;
a memory comprising:
a pre-trained artificial intelligence algorithm which upon execution by a processor is configured to implement the steps comprising:
generate a dynamic face model of the face of the user, the dynamic face model comprises information related to positioning of the plurality of electrodes, the stimulation pattern for the plurality of electrodes, and stimulation parameters for each electrode of the plurality of electrodes, wherein the dynamic face model is stored in the memory,
wherein the dynamic face model upon execution by the processor configured to generate a stimulation pattern which when delivered by the stimulator to the target facial muscles induces a predetermined facial expression.
